(19) United States
(12) Patent Application Publication
(10) Pub. No.: US 2022/0199207 A1
(43) Pub. Date: Jun. 23, 2022

(54) System and Method for Facilitating Data Processing of Physiological Information

(71) Applicant: Tricog Health Pte Ltd, Singapore (SG)

(72) Inventors: Charit Charit, Bangalore (IN); Zainul Charbiwala, Bengaluru (IN); Udayan Dasgupta, Bangalore (IN); Abhinav Gujjar, Bangalore (IN)

(21) Appl. No.: 17/604,143

(22) PCT Filed: Apr. 15, 2020
(86) PCT No.: PCT/SG2020/050235

§ 371 Date: Oct. 15, 2021

(30) Foreign Application Priority Data
Apr. 16, 2019 (IN) 201941015295

(51) Int. Cl. G16H 10/60; G16H 50/70; A61B 5/00; H04L 67/55

(52) U.S. Cl. CPC G16H 10/60; G16H 50/70; A61B 5/7264; H04L 67/26; A61B 5/7232; A61B 5/0022; A61B 5/7221

(57) ABSTRACT

A system and method for facilitating data processing of physiological information is disclosed. The system for facilitating data processing of physiological information includes: a device operable to collect a dataset of the physiological information from a person; and a server operable to receive the dataset from the device, and classify the dataset into at least one preliminary group of medical condition. The server is arranged in data communication to send the preliminary group to a third party device for verification. The server is operable to receive a verified group from the third party device, and modify at least a part of algorithm associated with the classification based on the verified group.

### CROSS-REFERENCE TO RELATED APPLICATIONS

This application is the United States national phase of International Application No. PCT/SG2020/050235 filed Apr. 15, 2020, and claims priority to Indian Patent Application No. 201941015295 filed Apr. 16, 2019, the disclosures of which are hereby incorporated by reference in their entirety.

### BACKGROUND OF THE INVENTION

### Field of the Invention

The present invention relates to a system and method for facilitating data processing of physiological information.

### Description of Related Art

The following discussion of the background to the invention is intended to facilitate an understanding of the present invention only. It may be appreciated that the discussion is not an acknowledgement or admission that any of the material referred to was published, known or part of the common general knowledge of the person skilled in the art in any jurisdiction as at the priority date of the invention.

Heart diseases, for example cardiovascular diseases, are one of main causes of death in many countries. The heart diseases may occur depending on various factors such as ageing, lifestyles, poor eating habits and/or stress. The heart diseases may occur not only under-privileged people, but also urban and economically well-off people.

Although hospitals have adequate facilities and expertise for treatment of the heart diseases, a timely diagnosis of the heart diseases remains a problem. Because, such heart diseases may often occur an emergency event which requires a treatment within a golden hour and causes death or irreparable damage to a person's body if it is not treated within the golden hour.

The timely diagnosis of the heart diseases, via electrocardiography (ECG) machines, may be the first step to address the above problem. However, although the ECG machines have become relatively common, an accurate ECG analysis still requires the expertise of doctors, for example cardiac specialists.

Conventionally, to address the above problem, some people have used the ECG machines to take the ECG test, and sent the ECG record to the doctors for his diagnosis. However, the diagnosis has often been delayed, for example for few days, due to the non-availability of such doctors on-site at all times.

Others have used the ECG machines to take the ECG test, and sent images of the ECG record to the doctors via email or message for his early diagnosis. However, the diagnosis has still been delayed, for example for few hours, as the doctors may be in a procedure or is unavailable. In addition, the diagnosis may be inaccurate due to the quality of the images.

Others have used high-end ECG machines having in-built ECG analysis algorithms. Apart from such ECG machines being more expensive, the in-built ECG analysis algorithms have limited accuracy. For example, such ECG machines may not be comprehensive in clinical usage. In addition, such ECG machines typically require a specialized medical practitioner, such as a trained physician or nurse, to operate.

In light of the above, there exists a need for a systematic way of delivering an accurate data processing associated with a medical condition in a timely manner. There exists a further need to provide a solution that meets the mentioned needs or alleviates the challenges at least in part.

### SUMMARY OF INVENTION

The primary object of the invention is to provide a system for facilitating data processing of physiological information.

Another object of the invention is to provide a system for classifying physiological condition into a preliminary group of medical conditions.

Yet another object of the invention is to provide a verification system for verifying an accurate classification of physiological information into a preliminary group of medical conditions.

### BRIEF DESCRIPTION OF THE DRAWINGS

The present invention will now be described, by way of example only, with reference to the accompanying drawings, in which:

FIG. 1 shows a block diagram in accordance with some embodiments of the present invention.

FIG. 2 shows a flow diagram in accordance with some embodiments of the present invention.

FIG. 3 shows another block diagram in accordance with some embodiments of the present invention.

FIGS. 4 to 6 show examples of a display of information provided by a server in accordance with some embodiments of the present invention.

FIGS. 7(a)-7(c) shows examples of messages in accordance with some embodiments of the present invention.

Other arrangements of the invention are possible and, consequently, the accompanying drawings are not to be understood as superseding the generality of the preceding description of the invention.

### DESCRIPTION OF THE INVENTION

The present invention seeks to provide a system and method that addresses the aforementioned need at least in part.

Throughout the specification, unless the context requires otherwise, the word “comprise” or variations such as “comprises” or “comprising”, will be understood to imply the inclusion of a stated integer or group of integers but not the exclusion of any other integer or group of integers.

Furthermore, throughout the specification, unless the context requires otherwise, the word “include” or variations such as “includes” or “including”, will be understood to imply the inclusion of a stated integer or group of integers but not the exclusion of any other integer or group of integers.

A systematic way of delivering an accurate diagnosis in a timely manner is envisaged.

The technical solution may be in the form of a system and method for facilitating data processing of physiological information. In particular, a device may collect a dataset, for example a set of ECG data, associated with a medical condition from a person. A server may receive the dataset from the device, classify the dataset into at least one preliminary group of medical condition, and send the preliminary group to a third party device, for example a doctor's device such as cardiac specialist's device, for verification. The server may then receive a verified group from the third party device, and modify at least a part of algorithm associated with the classification based on the verified group. The modified algorithm may be used for future classification.

In this manner, the server can provide an accurate classification using the modified algorithm which is based on previous verification of the third party. In addition, the server can provide the classification in a timely manner, since the server can receive the set of ECG data in real-time or near real-time.

In one aspect, there is a system for facilitating data processing of physiological information comprising: a device operable to collect a dataset of the physiological information from a person; and a server operable to receive the dataset from the device, and classify the dataset into at least one preliminary group of medical condition, the server arranged in data communication to send the preliminary group to a third party device for verification, wherein the server is operable to receive a verified group from the third party device, and modify at least a part of algorithm associated with the classification based on the verified group.

In some embodiments, the algorithm is operable to learn or adapt based on the dataset as an input and the verified group as an output, to modify the at least a part of algorithm.

In some embodiments, the at least a part of algorithm includes at least one of the following: weight, link, function and parameter associated with the algorithm.

In some embodiments, the classification of the dataset includes a comparison between the dataset and the parameter.

In some embodiments, the classification of the dataset includes a decision of the preliminary group of the medical condition based on the comparison between the dataset and the parameter.

In some embodiments, if the preliminary group differs from the verified group, the server is operable to modify the parameter associated with the classification.

In some embodiments, the server is operable to determine a confidence level of the decision of the preliminary group and send the confidence level to the third party device.

In some embodiments, if the confidence level is lower than a predetermined confidence level, the server is operable to highlight the confidence level.

In some embodiments, the classification of the dataset includes an extraction of a predetermined condition based on the preliminary group, and the server sends the extracted predetermined condition to the third party device.

In some embodiments, the server is operable to highlight the extracted predetermined condition.

In some embodiments, the server is operable to alert the third party device to the extracted predetermined condition.

In some embodiments, the server sends the dataset with an annotation including rationale of the decision of the preliminary group of the medical condition to the third party device.

In some embodiments, the server is operable to generate a voice signal of the annotation, and the server sends the voice signal of the annotation to the third party device.

In some embodiments, the server is operable to select the third party device among a plurality of third parties' devices based on at least one of the following: the number of available third parties, ability of the third parties, person information or the preliminary group.

In some embodiments, the server is operable to send the dataset to the third party device first, and then send the preliminary group to the third party device after a predetermined time.

In some embodiments, the predetermined time is determined based on at least one of the following: ability of the selected third party device, the person information or the preliminary group.

In some embodiments, the server is operable to generate a report based on the preliminary group and the verified group, and send the report to at least one of the person or another third party device via a message and/or a push notification.

In some embodiments, the server is operable to compress and encrypt the dataset, and send the compressed and encrypted dataset to the third party device.

In some embodiments, the server is further operable to modify the at least a part of algorithm based on person information.

In some embodiments, the server is a cloud server.

In some embodiments, the third party includes a doctor.

In another aspect, there is a method for facilitating data processing of physiological information comprising: collecting, at a device, a dataset of the physiological information from a person; receiving, at a server, the dataset from the device; classifying, at the server, the dataset into at least one preliminary group of medical condition; sending, at the server, the preliminary group to a third party device for verification; receiving, at the server, a verified group from the third party device; and modifying, at the server, at least a part of algorithm associated with the classification based on the verified group.

In another aspect, there is a system for facilitating data processing of physiological information comprising: a device operable to collect a dataset of the physiological information from a person; and a server operable to receive the dataset from the device, and classify the dataset into at least one preliminary group of medical condition, the server arranged in data communication to send the preliminary group to a third party device for verification, wherein the server is operable to receive a verified group from the third party device, and modify at least one parameter associated with the classification based on the verified group.

In another aspect, there is a method for facilitating data processing of physiological information comprising: collecting, at a device, a dataset of the physiological information from a person; receiving, at a server, the dataset from the device; classifying, at the server, the dataset into at least one preliminary group of medical condition; sending, at the server, the preliminary group to a third party device for verification; receiving, at the server, a verified group from the third party device; and modifying, at the server, at least one parameter associated with the classification based on the verified group.

In another aspect, there is a system for aiding a diagnosis for a patient comprising: a device operable to collect data associated with a medical condition from the patient; and a server operable to receive the data from the device, analyse the data using at least one algorithm to generate information associated with the medical condition, and provide the information to a third party for determination of the diagnosis, wherein the server is operable to receive the determination of the diagnosis from the third party, and modify the algorithm based on the determination of the diagnosis.

In another aspect, there is a method for aiding a diagnosis for a patient comprising: collecting, at a device, data associated with a medical condition from the patient; receiving, at a server, the data from the device; analyzing, at the server, the data using at least one algorithm to generate information associated with the medical condition; providing, at the server, the information to a third party for determination of the diagnosis; receiving, at the server, the determination of the diagnosis from the third party; and modifying, at the server, the algorithm based on the determination of the diagnosis.

Other aspects of the invention may be apparent to those of ordinary skilled in the art upon review of the following description of specific embodiments of the invention in conjunction with the accompanying drawings.

The embodiments herein, the various features and advantageous details thereof are explained more fully with reference to the non-limiting embodiments that are illustrated in the accompanying drawings and /or detailed in the following description. Descriptions of well-known components and processing techniques are omitted to not unnecessarily obscure the embodiments herein. The examples used herein are intended merely to facilitate an understanding of ways in which the embodiments herein may be practiced and to further enable those of skilled in the art to practice the embodiments herein. Accordingly, the examples should not be construed as limiting the scope of the embodiments or invention herein.

Throughout the description, a communication device may include, but not be limited to, smartphone, desktop computer, laptop, tablet computer and wearable device, in particular intelligent wearable device such as smart watch, smart glasses or mobile virtual reality headset.

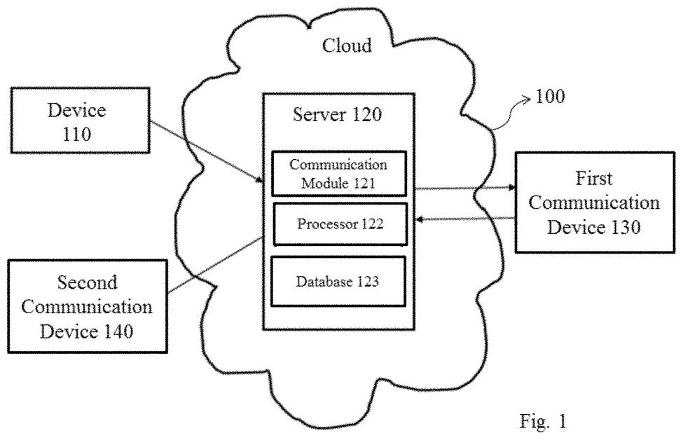

FIG. 1 shows a block diagram in accordance with some embodiments of the present invention. The system 100 may comprise a device 110 and a server 120.

The device 110 may include, but is not limited to, a home medical equipment adapted for residential use (i.e. for a person usage at home or other residential facilities). The home medical equipment may include, but not be limited to, an electrocardiography (ECG) machine which measures an electrical activity of the person's heart to show whether the heart is working normally.

The ECG machine may collect a dataset associated with the rhythm and activity of the person's heart and send the dataset to the server 120. The person or individual may include a human being and/or mammalian animal. For example, the dataset may be a set of data, for example ECG data. In some embodiments, the ECG machine may output the collected dataset on a screen and/or a paper. In some embodiments, the collected data may include, but is not limited to, SpO2 measurements, weight, blood pressure (BP), sugar levels, etc.

It is appreciable that in some embodiments the device 110 may be adapted for clinical and/or hospital use.

In some embodiments, the device 110 may include a communication device, for example the person's communication device. The communication device as the device 110 may collect the dataset associated with the rhythm and activity of the person's heart from the ECG machine, and send out the dataset to the server 120.

The server 120 may comprise a communication module 121, a processor 122 and a database 123. It may be appreciated that in some other embodiments, the communication module 121 and the processor 122 may be integrated. Some embodiments can be implemented in or supported by a cloud network infrastructure. The server 120 may be a cloud server which is built, hosted and delivered through a cloud computing platform over a communication network, for example Internet. The cloud server may be accessed remotely from a plurality of users including the person and the doctor.

The communication module 121 may include one or more modules or units which permit wired communications and/or wireless communications with the device 110 and/or other communication devices, for example first and/or second communication devices 130, 140 to be described below. For example, the communication module 121 receives ECG data from the device 110 and sends information generated by the processor 122 to the first and/or second communication device 130, 140. The information may include at least one preliminary group of medical condition determined by the processor 122 to be described below. The information may be in the form of audio signal, video signal, text signal, multimedia signals, or combination thereof. The information may also include various formats of data.

The processor 122 is operable to analyze the received dataset using the algorithm to decide the preliminary group of the medical condition, and generate the information including the preliminary group.

The system 100 may further comprise the at least one first communication device 130 used by a third party, for example a doctor such as a cardiac specialist. The doctor may be employed by the system 100 or contracted with the system 100 to provide the server 120 with a verified group of the medical condition.

After receiving the verification from the doctor via the communication module 121, the processor 122 is further operable to modify at least a part of algorithm based on the verification of the doctor.

The system 100 may further comprise the at least one second communication device 140 used by another party, for example person, guardian or family doctor. The person, guardian or family doctor may receive the verified group of the medical condition determined by the doctor from the server 120.

The database 123 is operable to store at least one of the following information: the dataset collected from the person, information generated by the processor 122, information verified by the doctor and a final report based on the verification.

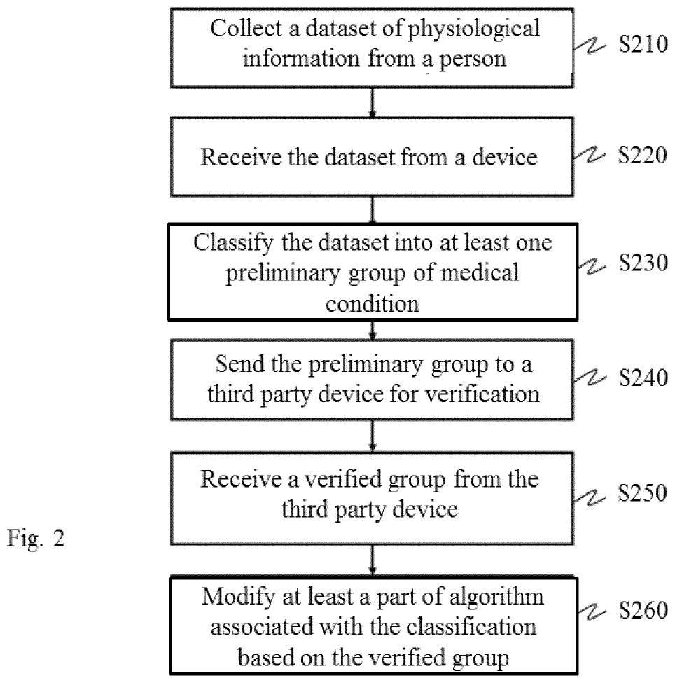

FIG. 2 shows a flow diagram in accordance with some embodiments of the present invention.

First, the device 110 may collect a dataset of physiological information from a person (S210). In some embodiments, the device 110 may detect the person's body and generate the dataset of the physiological information of the person. Thereafter, the device 110 may send the dataset to the server 120.

In some embodiments, the device 110 may continuously collect the dataset of the physiological information from the person, and send the dataset to the server 120 in real-time or near real-time.

The server 120 may then receive the dataset from the device 110 (S220). In some embodiments, the server 120 may receive the dataset from the device 110 in real-time or near real-time.

The server 120 may classify the dataset into at least one preliminary group of medical condition (S230).

In some embodiments, the server 120 may use at least one algorithm, for example a predetermined algorithm, and compare between the received dataset and at least one predetermined parameter using the predetermined algorithm. The server 120 may determine a preliminary group of the medical condition based on the comparison between the dataset and the predetermined parameter, to generate the information. The information may contain the preliminary group of the medical condition.

The server 120 may determine a confidence level of the decision of the preliminary group based on a difference value between the dataset and the predetermined parameter. The information may further contain the confidence level. If the confidence level is lower than a predetermined confidence level, the server 120 may highlight the confidence level in the information.

The classification of the dataset may include an extraction of a predetermined condition based on the preliminary group. The information may further contain the predetermined condition. The server 120 may highlight the extracted predetermined condition in the information. The server 120 may alert the third party, for example a doctor such as a cardiac specialist employed by the system 100 or contracted with the system 100, to the extracted predetermined condition, via a data communication to the third party device.

For example, if the server 120 determines that the preliminary group shows that an emergency event is likely to occur, the server 120 may inform the doctor in real-time or near real-time, with the alert.

In some embodiments, when the server 120 generates the information, the server 120 may include an annotation of the dataset, which relates to rationale of the decision of the preliminary group of the medical condition, in the information. Therefore, the doctor is able to easily understand or construe the preliminary group with respect to the dataset.

In some embodiments, the server 120 may generate a voice signal of the annotation, and include the voice signal of the annotation in the information. It may be appreciated that the voice signal may include a summary of the preliminary group.

In some embodiments, the server 120 may select at least one doctor among a plurality of doctors who are employed by the system 100 or contracted with the system 100. For example, the server 120 may select the doctor based on at least one of the following: the person information, the ability of the doctors, the preliminary group or the number of available doctors. The person information may include, but not be limited to, age, gender, weight, height, residence, medical history, etc. The ability of the doctors may include, but not be limited to, average processing time taken to determine or verify the medical condition, and experience.

For example, if the person's medical condition has been verified by a specific doctor, the server 120 may select the specific doctor for the subsequent verification of the medical condition. As another example, if the person is an infant, the server 120 may select a pediatrician among the doctors. If the server 120 determines that the preliminary group shows that an emergency event is likely to occur, the server 120 may select a highly experienced doctor among the doctors.

Although not shown, the server 120 may select two or more doctors among the plurality of doctors, so that the server 120 may obtain the verifications of the medical condition from the two or more doctors.

For example, the server 120 may select two or more doctors if there are large number of available doctors. As another example, if the server 120 determines that the preliminary group shows that an emergency event is likely to occur, the server 120 may select two or more doctors to obtain the verifications of the group from the doctors to improve accuracy. As another example, if the confidence level is lower than a predetermined confidence level, the server 120 may select two or more doctors to obtain the verifications of the medical condition from the doctors to improve accuracy.

After selecting the doctor, the server 120 may send the preliminary group to a third party device for verification (S240) and then receive a verified group from the third party device (S250). It may be appreciated that the server 120 may compress and/or encrypt the dataset and provide the compressed and/or encrypted dataset to the first communication device 130 used by the doctor. As described earlier, the information may contain the preliminary group determined by the server 120.

When the information is provided to the doctor, the server 120 may control the doctor's first communication device 130 to display the information, for example the preliminary group, in a desired manner. For example, the server 120 may provide the dataset to the first communication device 130 first, and then provide the information to the first communication device 130 after a predetermined time. Therefore, the first communication device 130 can display the dataset first, and subsequently display the information after the predetermined time.

As another example, the server 120 may provide the dataset and the information to the first communication device 130 together, but instruct the first communication device 130 to display the dataset first and subsequently display the information after the predetermined time. In some embodiments, the predetermined time may be determined based on at least one of the following: the person information, the ability of the selected doctor or the preliminary group.

For example, if it is required to pay attention to a specific person, the predetermined time may be increased, so that the doctor can classify a group of the medical condition based on the dataset, without prejudice. As another example, if the selected doctor has no previous experience, the predetermined time may be increased, so that the doctor can classify a group of the medical condition based on the dataset, without reliance on the preliminary group. As another example, if the server 120 determines that the preliminary group shows that an emergency event is likely to occur, the predetermined time may be increased, so that the doctor can classify a group of the medical condition based on the dataset, without prejudice.

Although not shown, the server 120 may generate a report based on the information including the preliminary group and the verified group. In some embodiments, the report may include the information and/or the verified group. In some embodiments, the report may be configured based on the recipient's preference or interest. For example, if the recipient is the person, the current medical condition based on the verified group of the medical condition may be highlighted. As another example, if the recipient is the guardian, the explanation of the home treatment associated with the current medical condition may be highlighted. As another example, if the recipient is the family doctor, the dataset and the summary of the verified group of the medical condition may be highlighted.

The server 120 may then send the report to the at least one second communication device 140 used by the person, guardian or family doctor, via a message, an email and/or a push notification. It may be appreciated that the server 120 may compress and/or encrypt the report and provide the compressed and/or encrypted report to the second communication device 140.

Thereafter, the server 120 may modify at least a part of algorithm, for example at least a part of the predetermined algorithm, associated with the classification based on the verified group (S260). In neural networks, over successive iterations, the algorithm is operable to learn or adapt based on the dataset as an input and the verified group as an output. In some embodiments, the at least a part of algorithm, for example weight, link, function and/or parameter associated with the algorithm, can be modified accordingly. In some other embodiments, a whole algorithm can be modified, changed or replaced.

In some embodiments, after receiving the verified group of the medical condition from the doctor, the server 120 may operate a machine learning algorithm by taking the dataset as an input and the verified group as an output, so that the machine learning algorithm modifies the predetermined algorithm to output the verified group using the dataset as the input. The at least a part of the predetermined algorithm may be modified in order to output the verified group as a new output. In this manner, the at least a part of algorithm used for the classification can be trained automatically, without the user's input.

It may be appreciated that in some embodiments, one algorithm may be modified based on the verified group. In some other embodiments, two or more algorithms may be modified based on the verified group. If there are a plurality of possible modifications, the server 120 may choose at least one modification. For example, the server 120 may simulate the modifications by taking another dataset as an input and another verified group as an output, and choose at least one modification which can output another verified group.

In some embodiments, the server 120 may modify the parameter, as a part of algorithm, which is used to compare the dataset to determine the preliminary group, based on the verified group. In some embodiments, if the preliminary group differs from the verified group, the server 120 may modify the parameter associated with the classification.

For example, if the preliminary group has been determined to be “malignant” based on the comparison the dataset to the parameter, but the verified group has been determined to be “benign”, the server 120 may modify the parameter to be increased, according to the doctor's verification. In this manner, the server 120 will be able to compare new dataset with the modified parameter to determine the preliminary group for the new dataset.

In another example, if the preliminary group has been determined to be “benign” with a low confidence level, and the verified group has been determined to be “benign”, the server 120 may modify the parameter accordingly. In this manner, the server 120 will be able to determine the preliminary group for the same dataset as “benign”, with a high confidence level, in future.

In some embodiments, the server 120 may further modify the at least a part of algorithm associated with the classification based on the person's information. For example, if the weight of the person has been increased, the server 120 may modify the parameter, as a part of algorithm, to be in line with the increased weight.

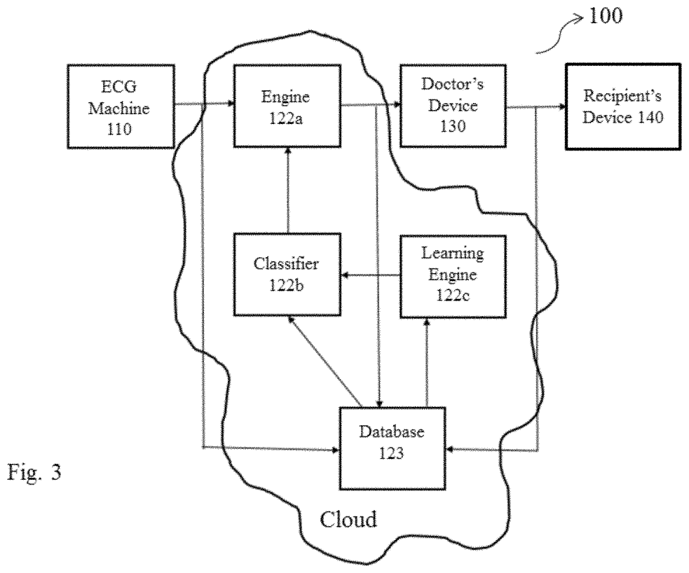

FIG. 3 shows another block diagram in accordance with some embodiments of the present invention.

The device 110 may comprise an ECG machine 110. The ECG machine 110 may comprise a communication module (not shown) which allows a seamless transmission of the ECG dataset to the server 120, for example a cloud server.

In some embodiments, the communication module may be pre-configured to push raw ECG dataset, for example, voltage points as opposed to scans, to the server 120 via a communication network, for example 2G, 3G or Wi-Fi. In this manner, the transmission requirements can be kept low, while high fidelity images can be created at any later time if necessary.

In some embodiments, the communication module may be designed to transmit the ECG dataset to the server 120 without any change of an explicit workflow. The ECG data can be transmitted whenever the person or user requests. For example, when the person selects a button of “Print ECG” or “Capture ECG”, the ECG data can be transmitted to the server 120. As another example, when the ECG capture is verified by a trained personnel assisting the person, the ECG data can be transmitted to the server 120. Verification may be performed via visual inspection or by an image capturing device which operates to capture an image of the ECG data for analysis.

In some embodiments, the communication module may be designed to report operational issues automatically, thereby enabling the remote diagnosis or assessment of the functionality of the device 110 and/or communication link between the device 110 and server 120.

An example of such remote diagnosis includes arranging the device 110 to send multiple signals (also known as ‘heartbeats’) to the server 120 at every predetermined interval for onward transmission to a computing device (not shown) which analyzes these signal. In some embodiment, one or more of the multiple signals can include useful information for further analysis by the server 120. For example, the useful information can be embedded in one or more of the multiple signals. Such embedded information can include information about key system parameters like strength of cellular signal, remaining battery charge and other information helping in remote debugging. If the battery levels indicate a decreasing trend before the heartbeats stop completely, it can be concluded that the device 110 needs to be charged. If the cellular signal strength keeps fluctuating, it can be concluded that there are network problems at the remote location. Based on the debug information received in the heartbeats, malfunction of the device 110 can be detected. In this manner, the user, for example the person, can notice the malfunction of the device 110 and repair the device 110 properly.

In some embodiments, the predetermined interval is 1 minute.

In some embodiments, the computing device can be located within the communication module or the sever 120. In other embodiments, the computing device can be located remote to the server 120 in another communication device (not shown). In yet another embodiment, the computing device can be located in the first communication device 130 or the second communication device 140.

In some embodiments, the communication module may implement compression and/or encryption techniques for the secure transmission of the ECG data, even with low bandwidth protocols, for example 2G.

As described above, the server 120 may comprise the communication module 121, the processor 122 and the database 123. The processor 122 may comprise an engine 122a, a classifier 122b and a learning engine 122c.

Some embodiments can be implemented in or supported by a cloud network infrastructure. The server 120 may be a cloud server which is built, hosted and delivered through a cloud computing platform over a communication network. The cloud network infrastructure may be shared by a set of remote clinics or hospitals.

In some embodiments, based on the remote site from where the ECG data is received, the server 120 may select an organization where doctors belong to, and route the ECG data to the organization.

In some embodiments, within the organization, the ECG data may be scheduled to be pushed automatically, based on a set of rules, to a set of doctors. The rules may allow to schedule an appropriate doctor for the quick processing of the ECG data. The rules may include, but not be limited to the following:

The number of available doctors at that time, their list of pending ECG diagnosis, their average processing time, etc.

Time when the ECG data was received (for example, ECG data received earlier may be processed earlier)

Quality Service Levels (for example, response time) required by a person (for example, diagnostic centers may be able to tolerate larger diagnosis delays than individual clinics)

Criticality of the ECG data based on preliminary group (for example, the critical cases may be scheduled ahead of normal cases).

In some embodiments, a two-step verification may be used. For example, after the doctor verifies the preliminary group determined by the server 120, the doctor's verification may be provided to another doctor, for example a person nominated physician, for a further verification. If the physician does not confirm the doctor's verification with a predetermined time, the unconfirmed verification (i.e. the doctor's verification) may be sent out to the recipient's device 140. Thereafter, the confirmed verification by the physician may be provided when it is available.

The classifier 122b may use the algorithm when the person's dataset is compared to the parameter to determine the preliminary group. The algorithm may allow the engine 122a to determine the preliminary group for at least a part of medical conditions, for example cardiac conditions, which are evident in the ECG data. To hasten a verification, each preliminary group may be linked to a series of measurements and/or evidences on the raw ECG data and an averaged component beat for each lead. In this manner, understandable features may be picked up by the algorithm to justify the preliminary group.

In some embodiments, the algorithm may allow the engine 122a to determine a confidence level of the preliminary group based on a difference value between the dataset and the parameter, so that the doctor can focus on the verification for the dataset which has a low confidence level. It is appreciable that the confidence level may be derived via other algorithms, such as, but not limited to, machine learning algorithms.

The engine 122a may classify the collected dataset into at least one preliminary group of the medical condition using the parameter and/or the algorithm and generate the information such as the preliminary group, and thereby improve the efficiency of the doctor. When the preliminary group is incorrect and thereby the parameter and/or algorithm need to be updated or modified, the doctor may provide a verified group before sending the report to the recipient's device 140, for example person's, guardian's or family doctor's device.

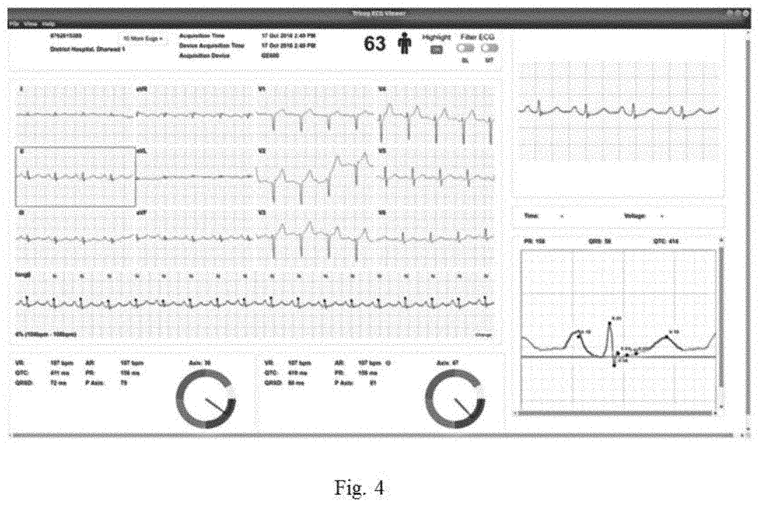

Therefore, the recipient's device 140 can obtain an accurate and verified report in a timely manner. In the meantime, the parameter and/or algorithm learn from the verified group and improve accordingly by the learning engine 122c. This virtuous cycle allows the parameter and/or algorithm to continuously improve and reduce the cognitive load and resource requirements on the doctor. FIGS. 4 to 6 show examples of a display of information provided by a server in accordance with some embodiments of the present invention.

The first communication device 130, for example doctor's communication device 130, may comprise at least one screen, for example single monitor or dual monitor. In some embodiments, the display of the screen may be controlled by the doctor's communication device 130. In some embodiments, the display of the screen may be controlled by the server 120.

In some embodiments, as shown in FIG. 4, the screen may display raw ECG data, for example raw ECG waveforms, for a predetermined time delay, before the preliminary group and/or measurements are displayed. The predetermined delay is deliberately included so as to minimize one or more cognitive biases arising from the preliminary group (when displayed on screen) from affecting the doctor's verified group.

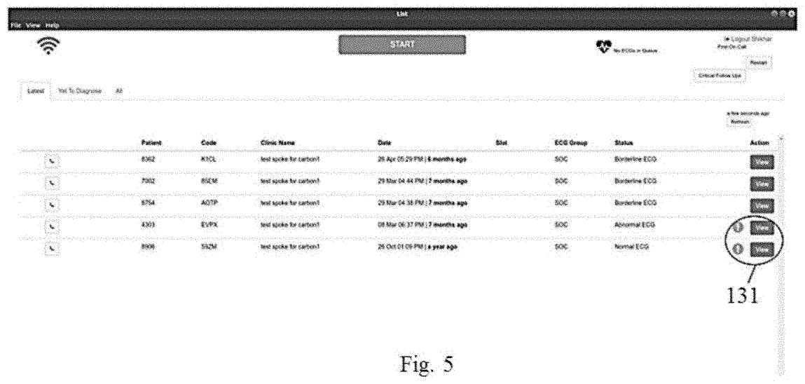

In some embodiments, the server 120 may highlight the certain preliminary group in the screen. For example, as shown in FIG. 5, if the server 120 determines that the preliminary group shows that an emergency event is likely to occur, the server 120 may inform the doctor of the preliminary group with the alert 131.

In some embodiments, the server 120 can be configured to receive input signals from one or more third party doctor(s). The one or more third party doctor(s) can provide explicit or implicit input signals via at least one input devices including but not limited to keyboards, mouse and biometric devices. Such explicit and implicit inputs include keyboard strokes, mouse movements, eye movements (via cameras), biometric input etc. to improve the overall quality of verification and/or the speed of verification. In some embodiments, inputs entered by third party doctors may be fed to the algorithms to identify one or more input patterns which are then used to present better views of the results customized to the preference of the doctor(s).

As an example, input signals such as doctors' keyboard strokes, mouse movements, eye movements (via camera) can be sent to or obtained by the server 120 (via processor 122) to glean information about which aspects of the ECG analysis is taking him the most time, and which aspects or features is the doctor trying to figure out at a particular time. This information can be used to develop methods to present relevant information to the doctor customized to his needs. In some embodiments, the server 120 can be trained via learning engine 122c to understand which aspects of the ECG the doctor has focused on in order to arrive at the verification. It is appreciable that the learning engine 122c, classifier 122b may include another learning algorithm to correlate the input signals to one or more input patterns.

In another example, the doctor can correct annotations (like beat shapes, locations, etc.) which can be user to further improve the algorithms.

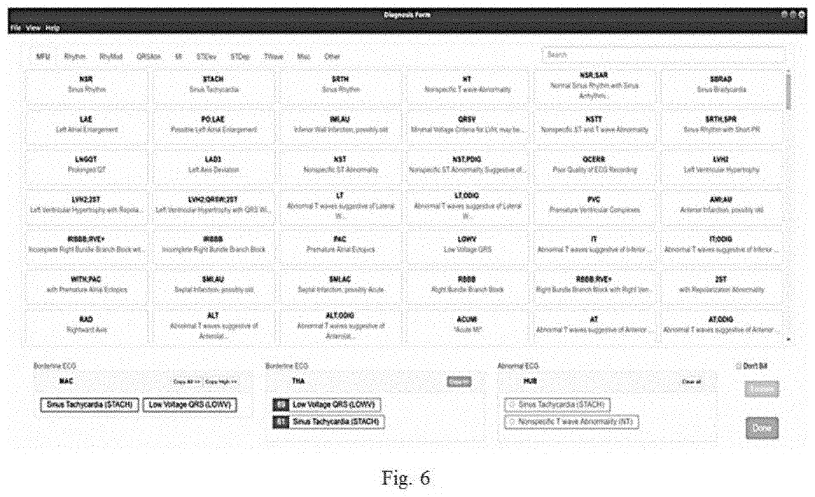

In some embodiments, as shown in FIG. 6, the user interface of the doctor's communication device 130 shows a list of possible conclusions. The doctor can provide explicit or implicit input signals by selection of at least one of the possible conclusions form displayed on the screen. It may be appreciated that the doctor can correct any entries of the list or create a new entry/list.

In yet another example, the parameter and/or the algorithm can use the feedback provided by the doctor to improve itself in various ways.

In yet another example, the ECG-doctor's verification pairs can be used to improve classifier models.

In yet another example, the ECG-annotation pairs (like beat shapes, Wave morphologies etc. for each ECG) can be used to improve classifier models.

In yet another example, the ECG-annotations-doctor's verification combinations can be used to tune parameter settings and improve decision rules.

In summary, the input signals such as key strokes, mouse movements and eye movements can be used to improve the viewer. For example, providing a plot of R-wave amplitudes for precordial leads may help doctors quickly see R-wave progression, a key ECG parameter. This would save them the trouble of viewing each of the six precordial leads sequentially and mentally comparing the amplitudes.

In some embodiments, the screen may display the preliminary group linked to underlying evidences (for example, lower level features and measurements on both the raw ECG waveforms and the averaged component beat). This may allow the doctor to verify the preliminary group quickly and easily.

In some embodiments, the doctor's communication device 130 may add automated voice based summary explaining the key steps (for example, rate, rhythm, wave morphologies, ST morphologies, etc.) taken to arrive at the preliminary group. This may allow the doctor to quickly understand the preliminary group.

In some embodiments, the doctor's communication device 130 may provide a haptic feedback to the doctor, if the server 120 determines that the person's dataset relates to a critical case.

In some embodiments, the doctor's communication device 130 may provide an inbuilt support of hierarchical cardiac conditions (for example, in the form of acronyms and/or synonyms) linked to the associated expanded explanations, to allow the doctor to quickly complete the verification with minimal typing or without typing. It is appreciable that the acronyms and/or synonyms may be used to auto-complete or assist-complete the verification.

In some embodiments, the doctor's communication device 130 may provide an in-viewer support for tools for cleaning up noise (for example, filters to eliminate baseline wander, muscle tremor), identifying the averaged component beat for each lead, identifying baseline for each lead, etc.

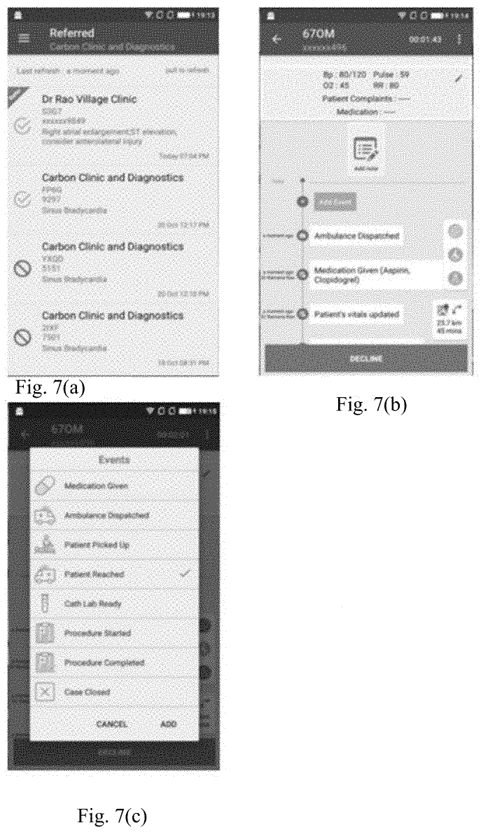

In some embodiments, the doctor's communication device 130 may provide an application (“App”) based ECG review system with tools for reviewing the ECG data and verifying the preliminary group, along with the associated tools to accelerate next steps of treatment, as shown in FIG. 7 below.

FIGS. 7(a)-7(c) shows examples of messages in accordance with some embodiments of the present invention.

Based on the recipient's preferences and use case, the ECG verification can be sent out to the recipient via a message, an email and/or an application. Apart from the remote clinic or hospital where the case was first registered, these messaging platforms can be extended to include ambulances and neighbouring hospitals, so as reduce door-to-balloon time and accelerate treatment.

It may be appreciated by the person skilled in the art that variations and combinations of features described above, not being alternatives or substitutes, may be combined to form yet further embodiments falling within the intended scope of the invention.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the scope of the embodiments as described herein.

1. A system for facilitating data processing of physiological information comprising:
a device operable to collect a dataset of the physiological information from a person; and
a server operable to receive the dataset from the device, and classify the dataset into at least one preliminary group of medical condition, the server arranged in data communication to send the preliminary group to a third party device for verification,
wherein the server is operable to receive a verified group from the third party device, and modify at least a part of algorithm associated with the classification based on the verified group.

2. The system according to claim 1, wherein the algorithm is operable to learn or adapt based on the dataset as an input and the verified group as an output, to modify the at least a part of algorithm.

3. The system according to claim 2, wherein the at least a part of algorithm comprises at least one of the following: weight, link, function and parameter associated with the algorithm.

4. The system according to claim 3, wherein the classification of the dataset comprises a comparison between the dataset and the parameter.

5. The system according to claim 4, wherein the classification of the dataset comprises a decision of the preliminary group of the medical condition based on the comparison between the dataset and the parameter.

6. The system according to claim 5, wherein if the preliminary group differs from the verified group, the server is operable to modify the parameter associated with the classification.

7. The system according to claim 5, wherein the server operable to determine a confidence level of the decision of the preliminary group and send the confidence level to the third party device.

8. The system according to claim 7, wherein if the confidence level is lower than a predetermined confidence level, the server is operable to highlight the confidence level.

9. The system according to claim 5, wherein the classification of the dataset comprises an extraction of a predetermined condition based on the preliminary group, and the server sends the extracted predetermined condition to the third party device.

10. The system according to claim 9, wherein the server is operable to highlight the extracted predetermined condition.

11. The system according to claim 9, wherein the server is operable to alert the third party device to the extracted predetermined condition.

12. The system according to claim 5, wherein the server sends the dataset with an annotation comprises rationale of the decision of the preliminary group of the medical condition to the third party device.

13. The system according to claim 12, wherein the server is operable to generate a voice signal of the annotation, and the server sends the voice signal of the annotation to the third party device.

14. The system according to claim 5, wherein the server is operable to select the third party device among a plurality of third parties' devices based on at least one of the following: the number of available third parties, ability of the third parties, person information or the preliminary group.

15. The system according to claim 14, wherein the server is operable to send the dataset to the third party device first, and then send the preliminary group to the third party device after a predetermined time.

16. The system according to claim 15, wherein the predetermined time is determined based on at least one of the following: ability of the selected third party device, the person information or the preliminary group.

17. The system according to claim 1, wherein the server is operable to generate a report based on the preliminary group and the verified group, and send the report to at least one of the person or another third party device via a message and/or a push notification.

18. The system according to claim 1, wherein the server is operable to compress and encrypt the dataset, and send the compressed and encrypted dataset to the third party device.

19. The system according to claim 4, wherein the server is further operable to modify the at least a part of algorithm based on person information.

20. The system according to claim 1, wherein the server is a cloud server.

21. The system according to claim 1, wherein the third party comprises a doctor.

22. A method for facilitating data processing of physiological information comprising:
collecting, at a device, a dataset of the physiological information from a person;
receiving, at a server, the dataset from the device;
classifying, at the server, the dataset into at least one preliminary group of medical condition;
sending, at the server-4 the preliminary group to a third party device for verification;
receiving, at the server, a verified group from the third party device; and
modifying, at the server, at least a part of algorithm associated with the classification based on the verified group.
